(19) United States
(12) Patent Application Publication
(10) Pub. No.: US 2013/0116787 A1
(43) Pub. Date: May 9, 2013

(54) ADJUSTABLE GRAFT ATTACHMENT

(71) Applicants: Michael Charles Ferragamo, Foster, RI (US); Michael James Perriello, Hopedale, MA (US); Alfred Rodrigue Berube, JR., North Attleboro, MA (US)

(72) Inventors: Michael Charles Ferragamo, Foster, RI (US); Michael James Perriello, Hopedale, MA (US); Alfred Rodrigue Berube, JR., North Attleboro, MA (US)

(73) Assignee: SMITH & NEPHEW, INC., Memphis, TN (US)

(21) Appl. No.: 13/291,522

(22) Filed: Nov. 8, 2011

(51) Int. Cl. A61F 2/08

(52) U.S. Cl. 623/13.14

(57) ABSTRACT

A graft attachment device includes a platform member, a ratchet mechanism, and a graft connecting element. The ratchet mechanism includes a rack member and a pawl member coupled to the rack member. The rack member has a first end, a second end, and teeth between the first end and the second end. The platform member is coupled to the first end. The pawl member is configured to engage with the teeth of the rack member such that movement of the pawl with respect to the rack member and the platform member is free in a first direction towards the first end of the rack member while being limited in a second direction towards the second end of the rack member. The second direction is opposite the first direction. The graft connecting element is coupled to the pawl member and configured for attachment to a graft.

### TECHNICAL FIELD

This document relates to graft attachment.

### BACKGROUND

An anterior cruciate ligament (ACL) that has ruptured and is non-repairable is generally replaced arthroscopically by a tissue graft. The tissue graft can be harvested from a portion of a patellar tendon having so called “bone blocks” at each end, and from the semitendinosus and gracilis. Alternatively, the tissue graft can be formed from synthetic materials or from a combination of synthetic and natural materials. The replacement tissue graft can be implanted by securing one end of the tissue graft in a socket formed in a passage within the femur, and passing the other end of the graft through a passage formed in the tibia.

### SUMMARY

According to one aspect, a graft attachment device includes a platform member, a ratchet mechanism, and a graft connecting element. The ratchet mechanism includes a rack member and a pawl member coupled to the rack member. The rack member has a first end, a second end, and teeth between the first end and the second end. The platform member is coupled to the first end. The pawl member is configured to engage with the teeth of the rack member such that movement of the pawl with respect to the rack member and the platform member is free in a first direction towards the first end of the rack member while being limited in a second direction towards the second end of the rack member. The second direction is opposite the first direction. The graft connecting element is coupled to the pawl member and configured for attachment to a graft.

Implementations of this aspect may include one or more of the following features.

For example, a first filament may be coupled to the second end of the rack member. A second filament may be coupled to the pawl member such that pulling the second filament while pulling the first filament results in the pawl member moving in the first direction. The first end of the rack member may include a pulley structure. The second filament may be coupled to the pawl member and the pulley structure such that pulling the second filament while pulling the first filament results in the pawl member moving in the first direction. The platform member may be pivotally coupled to the first end of the rack member. The platform member may be coupled to the first end of the rack member such that, when the platform member is in a first position, the rack member is positioned at a center of the platform member and, when the platform member is in a second position, the rack member is positioned at an end of the platform member. The pawl member may include a channel and a flange extending into the channel. The rack member may extend through the channel such that the flange engages the teeth of the rack member. The pawl member may define a tunnel. The graft connecting element may be a continuous loop that passes through the tunnel defined by the pawl member.

According to another aspect, a method for attaching a graft includes advancing a graft attachment device and graft through a bone tunnel, passing the platform member through an opening in the tunnel, positioning the platform member on a cortical layer of bone such that the pawl, graft connecting element, and graft are contained in tunnel, and moving the pawl along the rack member in the first direction such that the graft connecting element and graft move in the first direction. The graft attachment device includes a platform member and a ratchet mechanism. The ratchet mechanism includes a rack member coupled to the platform member, a pawl member coupled to the rack member, and a graft connecting element coupled to the pawl member. The rack member has teeth. The pawl member is configured to engage with the teeth of the rack member such that movement of the pawl with respect to the rack member and the platform member is free in a first direction towards a first end of the rack member while being limited in a second direction towards a second end of the rack member. The second direction is opposite the first direction. The graft connecting element is attached to a graft.

Implementations of this aspect may include one or more of the following features.

For example, advancing the graft attachment device can include pulling a filament attached to a first end of the platform member through the bone tunnel. An end of the rack member may be coupled to the platform member. Pulling the filament may include pulling the filament while the platform member is in a first position in which the end of the rack member is positioned at an end of the platform member. Positioning the platform member may include manipulating the platform member to a second position in which the first end of the rack member is positioned at a center of the platform member. Positioning the platform member may include manipulating the platform member such the platform member straddles the opening of the bone tunnel while the rack member extends from the platform member into the bone tunnel. An end of the rack member may be coupled to the platform member. Manipulating the platform member may include comprises manipulating the platform member such that the end of the rack member is positioned substantially at a center of the platform member while the platform member straddles the opening. Manipulating the platform member may include moving the platform from a first position in which an end of the rack member is positioned at an end of the platform member to a second position in which the end of the rack member is positioned at a center of the platform member. Moving the pawl along the rack member in the first direction may include pulling on a first filament coupled to the pawl while pulling on a second filament coupled to the rack member. The filament may be coupled to a pulley structure located at an end of the rack member.

The details of one or more implementations are set forth in the accompanying drawings and the description below. Other features, aspects, and advantages will become apparent from the description, the drawings, and the claims.

### BRIEF DESCRIPTION OF THE DRAWINGS

FIG. 1A is a perspective view of a graft attachment device implanted within a femur.

FIG. 1B is a perspective view of an alternative implantation of the graft attachment device within the femur.

FIG. 2 is another perspective view of the graft attachment device.

FIG. 3 is a perspective view of a rack member.

FIGS. 4A-4C are perspective and side views of a pawl member and a rack member.

FIG. 5 is a side view of a graft connecting element.

FIGS. 6A-6B are perspective views of a platform member and a rack member.

### DETAILED DESCRIPTION

This document describes an example of an attachment device to anchor tissue grafts. The attachment device can, for example, be used to fixate tissue grafts, such as soft tissue and bone-tendon grafts, in a tunnel within a cortical bone layer of a bone. In some implementations, a distance from the cortical bone layer to the tissue graft can be adjusted, for example, while the attachment device and the tissue graft are in the tunnel. In some cases, the distance can be reduced, for example, from 6 mm to 0 mm.

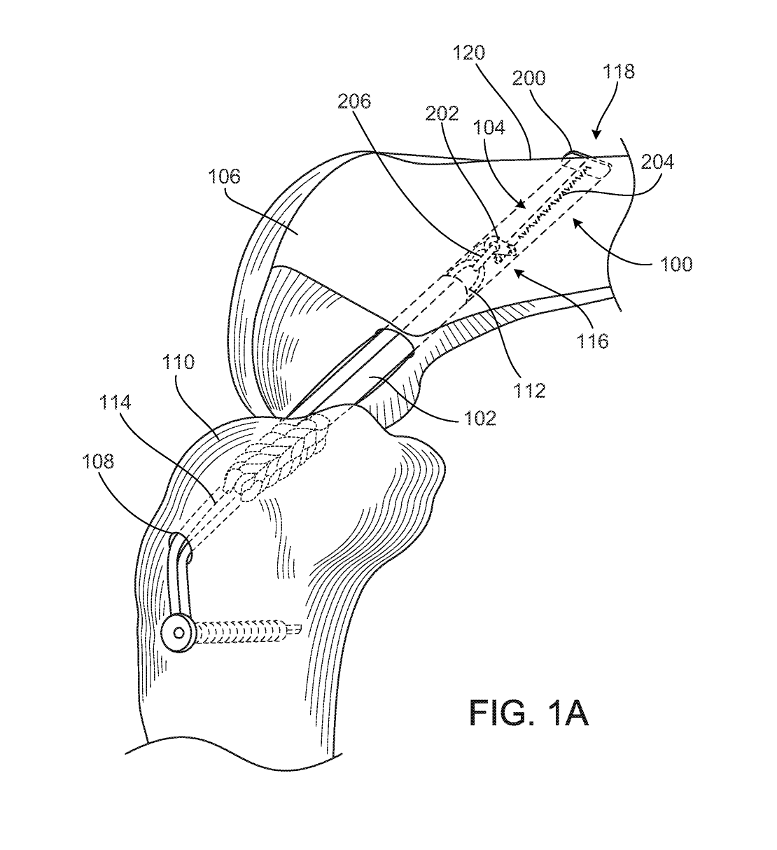

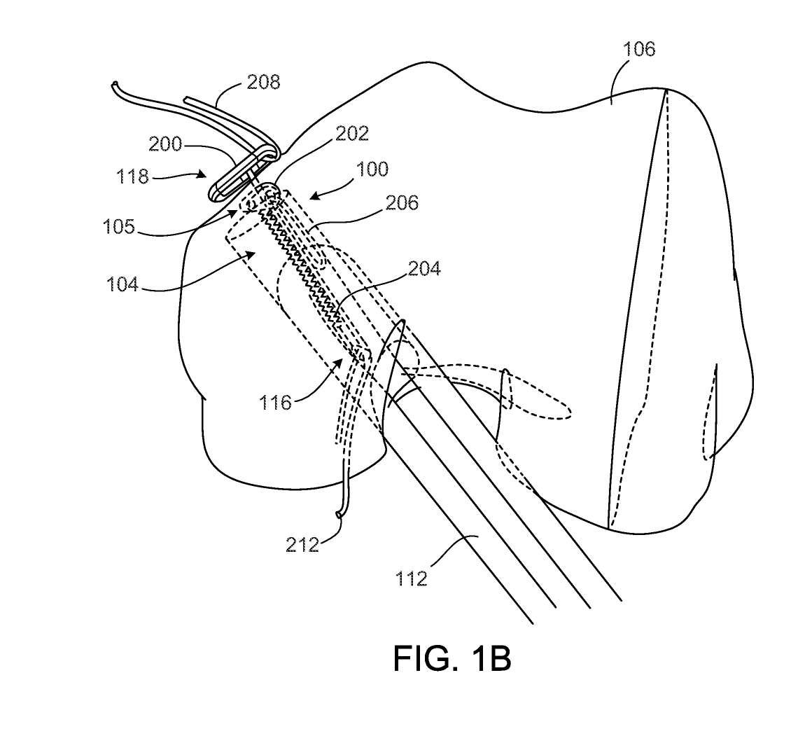

Referring to FIGS. 1A-1B, a graft attachment device 100 is implanted during, for example, an anterior cruciate ligament (ACL) repair and reconstruction procedure, and is adjustably attached to a graft 102 within a femoral tunnel 104. The femoral tunnel 104 can receive one end of the tissue graft 102 and is located within a femur 106. A tibial tunnel 108 can receive the other end of the tissue graft 102 and is located within a tibia 110. In some cases, one or both ends of the tissue graft 102 are attached to a filament, such as sutures 112, 114, for fixing the tissue graft 102 to the graft attachment device 100 or to other parts of the femur 106 and/or the tibia 110. A distal end 116 of the graft attachment device 100 can be secured to the tissue graft 102 while a proximal end 118 of the graft attachment device 100 can be secured to a cortical surface of bone 120. As illustrated in FIG. 1B, the femoral tunnel 104 can have a narrow tunnel portion 105.

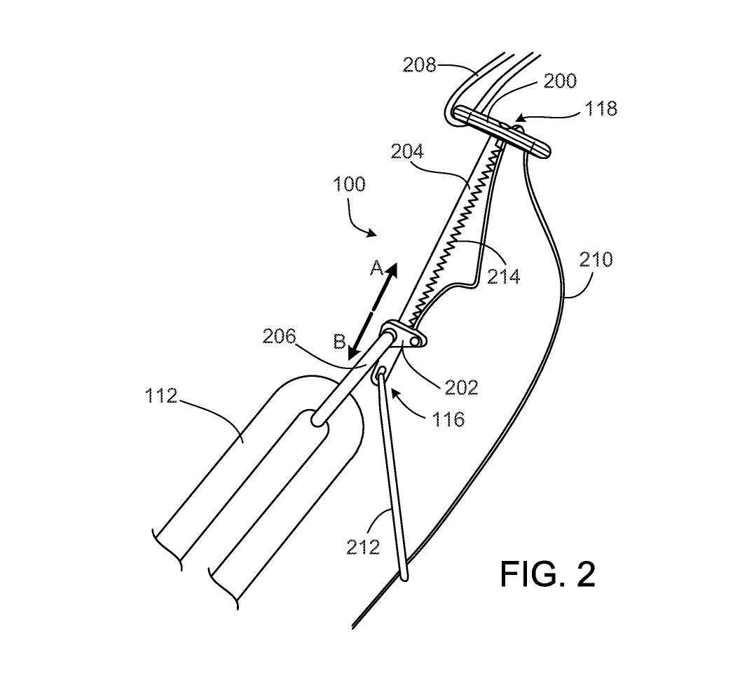

Referring also to FIG. 2, the graft attachment device 100 includes a platform member, such as button 200, that can be positioned on the cortical surface of bone 120. The device 100 also includes a ratchet mechanism, which includes a pawl member 202 that can be secured to the tissue graft 102 or to the suture 112, and a rack member 204 that connects the button 200 to the pawl member 202. The pawl member 202 and the rack member 204 cooperate to provide a one-way ratcheting action between the two members. In particular, the pawl member 202 is configured to engage with the teeth 214 of the rack member 204 such that movement of the pawl 202 with respect to the rack member 202 and the platform member 200 is free in a first direction, A, towards the proximal end of the rack member 204 while being limited in a second direction, B, towards the distal end of the rack member 204.

A graft connecting element 206 can connect the tissue graft 102 or the suture 112 to the pawl member 202. The graft connecting element 206 can be in the form of a continuous loop of suture or other material that is looped through an opening in the pawl member 202. Components of the graft attachment device 100 can be formed from any biocompatible material or a combination of biocompatible materials, such as certain metal alloys and polymers. Components of the graft attachment device 100 can include non-absorbable materials such as PEEK or Acetal. Alternatively, or additionally, components of the graft attachment device 100 can include bioabsorbable materials such as PLLA. A plurality of filaments, threads, sutures, or the like can be attached to portions of the graft attachment device 100 to help position and manipulate the graft attachment device 100 and the tissue graft 102 within the femoral tunnel 104. For example, a lead suture 208 can be looped through the button 200, a pull suture 210 can be looped through an opening in the pawl member 202, and a stabilizing suture 212 can be looped through an opening in the rack member 204.

In use, the lead suture 208 can be passed through the femoral tunnel 104 and pulled out through an opening on an anterior side of the femur 106 (FIGS. 1A-1B). A surgeon can then position the graft attachment device 100 and the proximal end of the attached tissue graft 102 within the femoral tunnel 104 by pulling on the lead suture 208. The lead suture 208 may be pulled until the button 200 of the graft attachment device 100 passes out of the femoral tunnel 104 and lies flat on the cortical surface of bone 120 of the femur 106. At this stage, the surgeon can adjust, as required, the distance between the cortical surface of bone 120 and the tissue graft 102 and/or the tightness of the tissue graft 102 within the femoral tunnel 104 by advancing the pawl member 202 along the rack member 204 towards the proximal end 118 of the graft attachment device 100. The distance between the cortical surface of bone 120 and the tissue graft 102 can be reduced by, for example, 0 to 20 mm.

The surgeon may advance the pawl member 202 along the rack member 204 in the first direction A by pulling on the pull suture 210. The pull suture 210 can be attached to the pawl member 202 such that the pull suture 210 pulls the pawl member 202 towards the proximal end 118. The stabilizing suture 212 can be attached to the rack member 204 and simultaneously pulled by the surgeon while pulling on the pull suture 210 such that the rack member 204 remains stationary within the femoral tunnel 104. The one-way ratcheting action between the pawl member 202 and the rack member 204, as described further below, prevents the pawl member 202 from moving in the second direction B along the rack member 204, towards the distal end 116 of the graft attachment device 100, thereby preventing the distance between the cortical surface of bone 120 and the tissue graft 102 from increasing. Alternatively, or additionally, the one-way ratcheting action between the pawl member 202 and the rack member 204 can discourage the tightness of the tissue graft 102 from decreasing.

A graft attachment device 100 installed within, for example, the femoral tunnel 104 can exhibit a longitudinal stiffness of around 140 N/mm to 335N/mm in a direction generally parallel to a length of the femoral tunnel 104. In a typical ACL application, the attached graft attachment device 100 will elongate by no more than, for example, around 0.5 mm during use. A distal end of the tissue graft 102 can be attached to the tibia 110 by using, for example, interference screws.

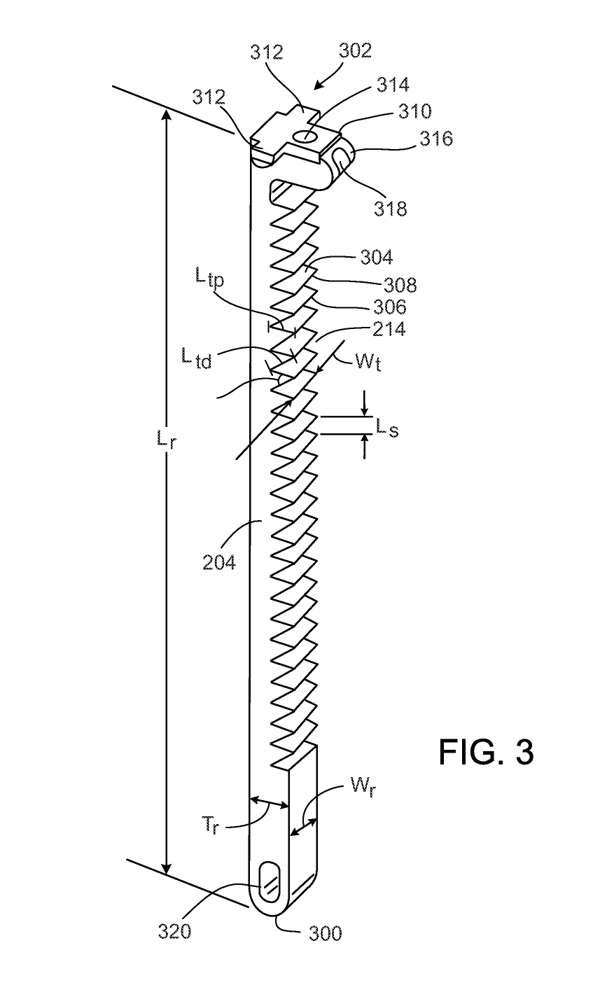

Referring to FIG. 3, the rack member 204 includes a plurality of teeth 214 positioned along a length (Lᵣ, for example 20 mm) of the rack member 204. The teeth 214 are shaped, sized, and spaced such that the pawl member 202 can engage with the teeth 214 to incrementally move from a distal end 300 of the rack member 204 to a proximal end 302 of the rack member 204 while being prevented from moving in the opposite direction. The specific design and dimensions of the teeth 214 can vary as required. In the example shown, each of the teeth 214 has a proximal face 304, a distal face 306, and an edge 308. A width (Wₜ) of the teeth 214 is substantially equal to a width (Wᵣ, for example 1.5 mm) of the rack member 204. The proximal face 304 of length Lₜₚ (for example 1 mm) is orthogonally transverse to the length Lᵣ of the rack member 204 while the distal face 306 of length L_{td} (for example 1.3 mm) is obliquely transverse to the length Lᵣ. The proximal face 304 forms an angle C (for example 40°) with the distal face 306, and adjacent edges 308 are spaced apart from each other by a length Lₛ (for example 0.9 mm). The rack member 204 has a lateral cross-section that is generally rectangular. Alternatively, the lateral cross-section of the rack member 204 can be round, T-shaped, H-shaped, or the like.

The proximal end 302 of the rack member 204 includes a pulley structure 310 and suspension posts 312. The pulley structure 310 can have a guide hole 314, a curved surface 316, and a guide channel 318, generally located on the curved surface 316. In use, a filament, such as the pull suture 210 (FIG. 2), can be connected to the pawl member 202 (FIG. 2), and a free end of the filament can then be inserted through a distal opening of the guide hole 314 and pulled down towards the distal end 300 through the guide channel 318. In such a configuration, pulling down of the free end of the filament by the surgeon will cause the pawl member 202 to be pulled up towards the proximal end 302 of the rack member 204. Other pulley configurations, for example one incorporating a pulley wheel, can be used. Alternatively, the free end of the filament can be passed through the guide hole 314 and subsequently pulled up away from the distal end 300 of the rack member 204. In such a configuration, pulling up of the free end of the filament away from the distal end 300 will cause the pawl member 202 to be pulled up towards the proximal end 302 of the rack member 204. A stabilizing hole 320 can be located near the distal end 300 of the rack member 204 for receiving the stabilizing suture 212.

The suspension posts 312 extend laterally beyond the width Wᵣ of the rack member 204 and are designed to pivotally couple to a corresponding feature, such as an indentation, of the button 200 (FIG. 2) as described further below. The suspension posts 312 are formed as a pair and are disposed on opposing sides of the rack member 204. Alternatively, the suspension posts can be oriented such that they extend laterally beyond a thickness Tᵣ of the rack member 204. The suspension posts 312 can be generally cylindrical or semi-cylindrical in shape.

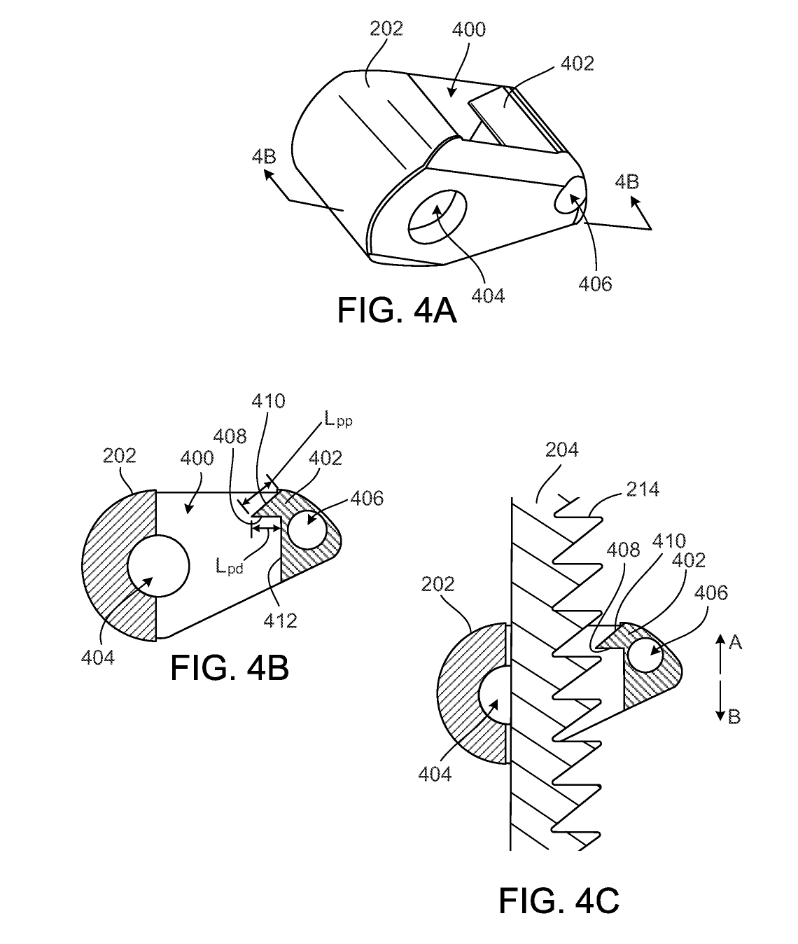

Referring to FIGS. 4A-4C, the pawl member 202 includes a channel 400, a pawl element 402 extending into the channel 400, a graft opening 404, and a suture opening 406. The channel 400 is sized and shaped to receive the distal end 300 of the rack member 204 such that the rack member 204 can generally move only along a direction parallel to its length Lᵣ. The pawl element 402 can be a flange or any structurally appropriate element that extends into the channel 400 to engage the teeth 214 of the rack member 204 disposed within the channel 400, as best seen in FIG. 4C. The pawl element 402 has a distal surface 408 and a proximal surface 410 for engaging, respectively, with the proximal face 304 and the distal face 306 of the teeth 214. The distal surface 408 and the proximal surface 410 of the pawl element 402 can have lengths of L_{pd} (for example 0.6 mm) and Lₚₚ (for example 0.75 mm), respectively, and are configured such that a force required to bend the pawl element 402 towards the proximal surface 410 is greater than a force required to bend the pawl element 402 towards the distal face 408. Additionally, or alternatively, a force required to bend the teeth 214 of the rack member 204 towards the distal face 306 can be greater than a force required to bend the teeth 214 towards the proximal face 304.

As shown in FIG. 4C, the pawl element 402 and the teeth 214 engage with each other such that the pawl member 202 can move relative to the rack member 204 in the first direction A while being limited from moving relative to the rack member 204 in the second direction B. The one-way ratcheting action between the pawl member 202 and the rack member 204 can depend on configurations of the pawl element 402, the teeth 214, or both. The pawl element 402 can be formed into an inner wall 412 of the channel 400. Alternatively, the pawl element 402 can be formed separately and attached to the inner wall 412.

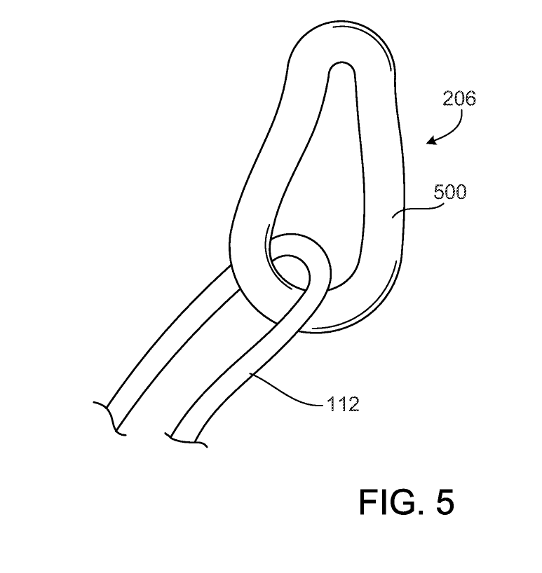

Referring to FIG. 5, a graft connecting element 206 can be looped through the graft opening 404, or tunnel, of the pawl member 202. The graft connecting element 206 can receive a suture 112 that is attached to the tissue graft 102 (FIG. 1A). Alternatively, the tissue graft 102 can attach directly to the graft connecting element 206. The graft connecting element 206 is generally a continuous loop and can be made from winding one or more loop filaments 500 through the graft opening 404.

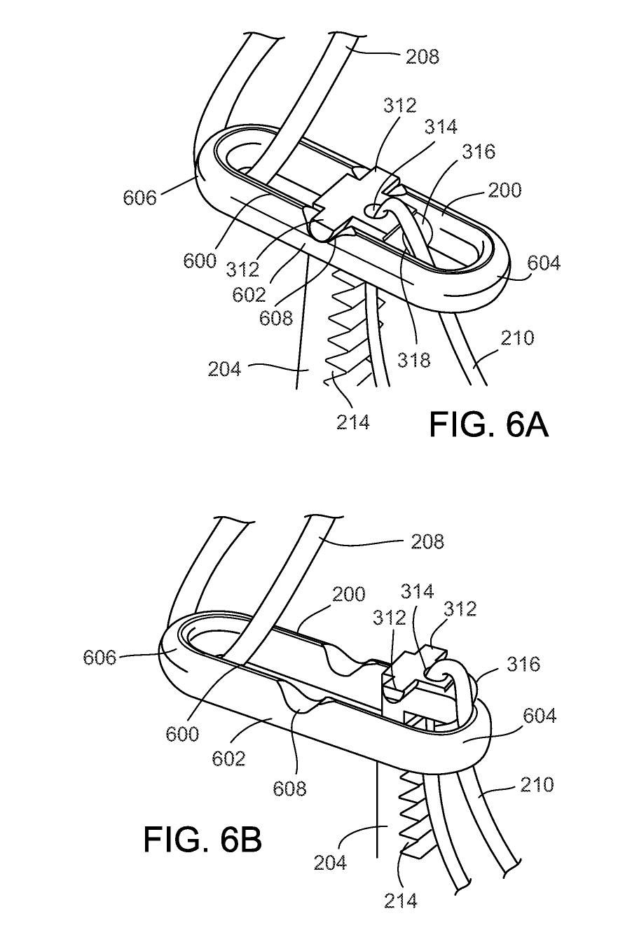

Referring to FIGS. 6A-6B, the suspension posts 312 of the rack member 204 can be positioned along an upper surface 600 of the button 200 such that the rack member 204 can slide freely between a center portion 602 of the button 200 and a trailing end 604. Additionally, the rack member 204 can slide towards a leading end 606 of the button 200. The center portion 602 includes a recessed portion 608 that is sized and shaped to pivotally engage the suspension posts 312. During positioning of the graft attachment device 100 and the tissue graft 102 within, for example, the femoral tunnel 104, the surgeon pulls on the lead suture 208 that is looped around the leading end 606 of the button 200, thereby causing the suspension posts 312 to become unseated from the recessed portion 608 and causing the rack member 204 to slide distally towards the trailing end 604 of the button 200, as shown in FIG. 6B. The button 200 also pivots at the trailing end 604 around the posts 312 so that a longitudinal axis of the button 200 becomes more parallel to a longitudinal axis of the rack member 204. This results in a reduction in the overall diameter of the device 100 relative to when the longitudinal axis of the button 200 is relatively more perpendicular to the longitudinal axis of the rack member 204. This may allow the device 100 to be pulled through a smaller diameter hole. Allowing the rack member 204 to slide to one end may provide for a reduction in the overall diameter that is great than can be achieved by simply allowing the button 200 to rotate about the suspension posts 312 while the posts 312 are located in the recessed portion 608 in the middle of the button 200.

After the button 200 passes out of the femoral tunnel 104 and lies flat on the cortical surface of bone 120 thereby straddling the opening, for example, on the anterior side of the femur 106, the rack member 204 may initially remain in the trailing end 604 as seen in FIG. 6B. Because the attached graft 102 puts the entire graft attachment device 100 under tension, manipulating the button 200 using sutures 208, 210, 212 will cause the suspension posts 312 to slide into the recessed portion 608 and become pivotally coupled to the button 200 at the center portion 602. Additionally, the upper surface 600 can be sloped towards the center portion 602 to help guide the suspension posts 312 towards the recessed portion 608.

While this document contains many specific implementation details, these should not be construed as limitations on the scope of any implementations or of what may be claimed, but rather as descriptions of features specific to particular implementations of particular implementations. Certain features that are described in this document in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. Thus, particular implementations of the subject matter have been described. Other implementations are within the scope of the following claims.

What is claimed is:

1. A graft attachment device comprising:
a platform member;
a ratchet mechanism comprising:
a rack member having a first end, a second end, and teeth between the first end and the second end, wherein the platform member is coupled to the first end;
a pawl member coupled to the rack member and configured to engage with the teeth of the rack member such that movement of the pawl with respect to the rack member and the platform member is free in a first direction towards the first end of the rack member while being limited in a second direction towards the second end of the rack member, the second direction being opposite the first direction; and
a graft connecting element coupled to the pawl member and configured for attachment to a graft.

2. The device of claim 1 further comprising:
a first filament coupled to the second end of the rack member; and
a second filament coupled to the pawl member such that pulling the second filament while pulling the first filament results in the pawl member moving in the first direction.

3. The device of claim 2 wherein:
the first end of the rack member includes a pulley structure; and
the second filament is coupled to the pawl member and the pulley structure such that pulling the second filament while pulling the first filament results in the pawl member moving in the first direction.

4. The device of claim 1 wherein the platform member is pivotally coupled to the first end of the rack member.

5. The device of claim 1 wherein the platform member is coupled to the first end of the rack member such that, when the platform member is in a first position, the rack member is positioned at a center of the platform member and, when the platform member is in a second position, the rack member is positioned at an end of the platform member.

6. The device of claim 1 wherein:
the pawl member includes a channel and a flange extending into the channel; and
the rack member extends through the channel such that the flange engages the teeth of the rack member.

7. The device of claim 1 wherein:
the pawl member defines a tunnel; and
the graft connecting element is a continuous loop that passes through the tunnel defined by the pawl member.

8. A method for attaching a graft, the method comprising:
advancing a graft attachment device and graft through a bone tunnel, the graft attachment device comprising:
a platform member;
a ratchet mechanism comprising:
a rack member coupled to the platform member and having teeth;
a pawl member coupled to the rack member and configured to engage with the teeth of the rack member such that movement of the pawl with respect to the rack member and the platform member is free in a first direction towards a first end of the rack member while being limited in a second direction towards a second end of the rack member, the second direction being opposite the first direction;
a graft connecting element coupled to the pawl member and attached to a graft;
passing the platform member through an opening in the tunnel;
positioning the platform member on a cortical layer of bone such that the pawl, graft connecting element, and graft are contained in tunnel; and
moving the pawl along the rack member in the first direction such that the graft connecting element and graft move in the first direction.

9. The method of claim 8 wherein advancing the graft attachment device comprises pulling a filament attached to a first end of the platform member through the bone tunnel.

10. The method of claim 9 wherein an end of the rack member is coupled to the platform member and pulling the filament comprises pulling the filament while the platform member is in a first position in which the end of the rack member is positioned at an end of the platform member.

11. The method of claim 8 wherein positioning the platform member comprises manipulating the platform member to a second position in which the first end of the rack member is positioned at a center of the platform member.

12. The method of claim 8 wherein positioning the platform member comprises manipulating the platform member such the platform member straddles the opening of the bone tunnel while the rack member extends from the platform member into the bone tunnel.

13. The method of claim 12 wherein an end of the rack member is coupled to the platform member and manipulating the platform member comprises manipulating the platform member such that the end of the rack member is positioned substantially at a center of the platform member while the platform member straddles the opening.

14. The method of claim 12 wherein manipulating the platform member comprises moving the platform from a first position in which an end of the rack member is positioned at an end of the platform member to a second position in which the end of the rack member is positioned at a center of the platform member.

15. The method of claim 8 wherein moving the pawl along the rack member in the first direction comprises pulling on a first filament coupled to the pawl while pulling on a second filament coupled to the rack member.

16. The method of claim 15 wherein the filament is coupled to a pulley structure located at an end of the rack member.
